(19) United States
(12) Patent Application Publication
(10) Pub. No.: US 2024/0366317 A1
(43) Pub. Date: Nov. 7, 2024

(54) APPARATUS FOR FLUID MANAGEMENT IN A ROBOTIC CATHETER-BASED PROCEDURE SYSTEM

(71) Applicant: Corindus, Inc., Newton, MA (US)

(72) Inventor: Andrew Clark, Arlington, MA (US)

(21) Appl. No.: 18/255,679

(22) PCT Filed: Jan. 14, 2021
(86) PCT No.: PCT/US2021/070040

§ 371 Date: Jun. 2, 2023

(51) Int. Cl. A61B 34/30; A61M 39/08

(52) U.S. Cl. CPC A61B 34/30; A61M 39/08; A61B 2034/301

(57) ABSTRACT

A cassette for use in a robotic drive of a catheter-based procedure system includes a housing configured to support a hemostasis valve having a base and a side port. The housing has a longitudinal device axis associated with an elongated medical device. The cassette also includes a first tube connection point positioned on the housing and above the longitudinal device axis. The first tube connection point is configured to receive a first tube. The cassette further includes a second tube connection point positioned proximate to a top edge of the housing and above the first tube connection point and the longitudinal device axis. The second tube connection point is configured to receive a second tube.

### FIELD

The present invention relates generally to the field of robotic medical procedure systems and, in particular, to an apparatus for managing fluid connections to an elongated medical device in a cassette in a robotic drive of a catheter-based procedure system.

### BACKGROUND

Catheters and other elongated medical devices (EMDs) may be used for minimally invasive medical procedures for the diagnosis and treatment of diseases of various vascular systems, including neurovascular intervention (NVI) also known as neurointerventional surgery, percutaneous coronary intervention (PCI) and peripheral vascular intervention (PVI). These procedures typically involve navigating a guidewire through the vasculature, and via the guidewire advancing a catheter to deliver therapy. The catheterization procedure starts by gaining access into the appropriate vessel, such as an artery or vein, with an introducer sheath using standard percutaneous techniques. Through the introducer sheath, a sheath or guide catheter is then advanced over a diagnostic guidewire to a primary location such as an internal carotid artery for NVI, a coronary ostium for PCI, or a superficial femoral artery for PVI. A guidewire suitable for the vasculature is then navigated through the sheath or guide catheter to a target location in the vasculature. In certain situations, such as in tortuous anatomy, a support catheter or microcatheter is inserted over the guidewire to assist in navigating the guidewire. The physician or operator may use an imaging system (e.g., fluoroscope) to obtain a cine with a contrast injection and select a fixed frame for use as a roadmap to navigate the guidewire or catheter to the target location, for example, a lesion. Contrast-enhanced images are also obtained while the physician delivers the guidewire or catheter so that the physician can verify that the device is moving along the correct path to the target location. While observing the anatomy using fluoroscopy, the physician manipulates the proximal end of the guidewire or catheter to direct the distal tip into the appropriate vessels toward the lesion or target anatomical location and avoid advancing into side branches.

Robotic catheter-based procedure systems have been developed that may be used to aid a physician in performing catheterization procedures such as, for example, NVI, PCI and PVI. Examples of NVI procedures include coil embolization of aneurysms, liquid embolization of arteriovenous malformations and mechanical thrombectomy of large vessel occlusions in the setting of acute ischemic stroke. In an NVI procedure, the physician uses a robotic system to gain target lesion access by controlling the manipulation of a neurovascular guidewire and microcatheter to deliver the therapy to restore normal blood flow. Target access is enabled by the sheath or guide catheter but may also require an intermediate catheter for more distal territory or to provide adequate support for the microcatheter and guidewire. The distal tip of a guidewire is navigated into, or past, the lesion depending on the type of lesion and treatment. For treating aneurysms, the microcatheter is advanced into the lesion and the guidewire is removed and several embolization coils are deployed into the aneurysm through the microcatheter and used to block blood flow into the aneurysm. For treating arteriovenous malformations, a liquid embolic is injected into the malformation via a microcatheter. Mechanical thrombectomy to treat vessel occlusions can be achieved either through aspiration and/or use of a stent retriever. Depending on the location of the clot, aspiration is either done through an aspiration catheter, or through a microcatheter for smaller arteries. Once the aspiration catheter is at the lesion, negative pressure is applied to remove the clot through the catheter. Alternatively, the clot can be removed by deploying a stent retriever through the microcatheter. Once the clot has integrated into the stent retriever, the clot is retrieved by retracting the stent retriever and microcatheter (or intermediate catheter) into the guide catheter.

In PCI, the physician uses a robotic system to gain lesion access by manipulating a coronary guidewire to deliver the therapy and restore normal blood flow. The access is enabled by seating a guide catheter in a coronary ostium. The distal tip of the guidewire is navigated past the lesion and, for complex anatomies, a microcatheter may be used to provide adequate support for the guidewire. The blood flow is restored by delivering and deploying a stent or balloon at the lesion. The lesion may need preparation prior to stenting, by either delivering a balloon for pre-dilation of the lesion, or by performing atherectomy using, for example, a laser or rotational atherectomy catheter and a balloon over the guidewire. Diagnostic imaging and physiological measurements may be performed to determine appropriate therapy by using imaging catheters or fractional flow reserve (FFR) measurements.

In PVI, the physician uses a robotic system to deliver the therapy and restore blood flow with techniques similar to NVI. The distal tip of the guidewire is navigated past the lesion and a microcatheter may be used to provide adequate support for the guidewire for complex anatomies. The blood flow is restored by delivering and deploying a stent or balloon to the lesion. As with PCI, lesion preparation and diagnostic imaging may be used as well.

When support at the distal end of a catheter or guidewire is needed, for example, to navigate tortuous or calcified vasculature, to reach distal anatomical locations, or to cross hard lesions, an over-the-wire (OTW) catheter or coaxial system is used. An OTW catheter has a lumen for the guidewire that extends the full length of the catheter. This provides a relatively stable system because the guidewire is supported along the whole length. This system, however, has some disadvantages, including higher friction, and longer overall length compared to rapid-exchange catheters (see below). Typically to remove or exchange an OTW catheter while maintaining the position of the indwelling guidewire, the exposed length (outside of the patient) of guidewire must be longer than the OTW catheter. A 300 cm long guidewire is typically sufficient for this purpose and is often referred to as an exchange length guidewire. Due to the length of the guidewire, two operators are needed to remove or exchange an OTW catheter. This becomes even more challenging if a triple coaxial, known in the art as a tri-axial system, is used (quadruple coaxial catheters have also been known to be used). However, due to its stability, an OTW system is often used in NVI and PVI procedures. On the other hand, PCI procedures often use rapid exchange (or monorail) catheters. The guidewire lumen in a rapid exchange catheter runs only through a distal section of the catheter, called the monorail or rapid exchange (RX) section. With a RX system, the operator manipulates the interventional devices parallel to each other (as opposed to with an OTW system, in which the devices are manipulated in a serial configuration), and the exposed length of guidewire only needs to be slightly longer than the RX section of the catheter. A rapid exchange length guidewire is typically 180-200 cm long. Given the shorter length guidewire and monorail, RX catheters can be exchanged by a single operator. However, RX catheters are often inadequate when more distal support is needed.

### SUMMARY

In accordance with an embodiment, a cassette for use in a robotic drive of a catheter-based procedure system includes a housing configured to support a hemostasis valve having a base and a side port. The housing has a longitudinal device axis associated with an elongated medical device. The cassette also includes a first tube connection point positioned on the housing and above the longitudinal device axis. The first tube connection point is configured to receive a first tube. The cassette further includes a second tube connection point positioned proximate to a top edge of the housing and above the first tube connection point and the longitudinal device axis. The second tube connection point is configured to receive a second tube.

In accordance with another embodiment, an apparatus for providing a fluid connection to a cassette for use in a robotic drive of a catheter-based procedure system includes a cassette housing and having a longitudinal device axis associated with an elongated medical device, a hemostasis valve positioned in the cassette housing. The hemostasis valve has a base and a side port. The apparatus further includes a first tube connection point positioned on the cassette housing and above the longitudinal device axis, a first tube coupled to the side port of the hemostasis valve and positioned in the first connection point, a valve having a plurality of ports wherein one of the plurality of ports is coupled to the first tube, a second tube connection point positioned proximate to a top edge of the cassette housing and above the first tube connection point and the longitudinal device axis, and a second tube coupled to one of the plurality of ports of the valve and positioned in the second tube connection point.

### BRIEF DESCRIPTION OF THE DRAWINGS

The invention will become more fully understood from the following detailed description, taken in conjunction with the accompanying drawings, wherein the reference numerals refer to like parts in which:

FIG. 1 is a perspective view of an exemplary catheter-based procedure system in accordance with an embodiment;

FIG. 2 is a schematic block diagram of an exemplary catheter-based procedure system in accordance with an embodiment;

FIG. 3 is a perspective view of a robotic drive for a catheter-based procedure system in accordance with an embodiment;

FIG. 4 is a diagram illustrating an elongated medical device axis of manipulation and the introductory point into the patient;

FIGS. 5a and 5b are diagrams illustrating the effect of the thickness of a robotic drive on the loss of working length;

FIG. 6 is a diagram illustrating an exemplary orientation to minimize loss of working length;

FIG. 7 is a perspective view of a device module with a vertically mounted cassette in accordance with an embodiment;

FIG. 8 is a rear perspective view of a device module with a vertically mounted cassette in accordance with an embodiment;

FIG. 9 is a front view of a distal end of a device module with a vertically mounted cassette in accordance with an embodiment;

FIG. 10 is a front view of a distal end of a device module with a horizontally mounted cassette in accordance with an embodiment;

FIG. 11 is a front view of a cassette including fluid management elements in accordance with an embodiment;

FIG. 12 is a front view of an apparatus for fluid management in accordance with an embodiment;

FIG. 13 is a front view of an apparatus for fluid management in accordance with an embodiment; and

FIG. 14 is a perspective view of a device module with a vertically amounted cassette and an apparatus for fluid management in accordance with an embodiment.

### DETAILED DESCRIPTION

The following definitions will be used herein. The term elongated medical device (EMD) refers to, but is not limited to, catheters (e.g. guide catheters, microcatheters, balloon/stent catheters), wire-based devices (guidewires, embolization coils, stent retrievers, etc.), and devices that have a combination of these. Wire-based EMD includes, but is not limited to, guidewires, microwires, a proximal pusher for embolization coils, stent retrievers, self-expanding stents, and flow divertors. Typically wire-based EMD's do not have a hub or handle at its proximal terminal end. In one embodiment the EMD is a catheter having a hub at a proximal end of the catheter and a flexible shaft extending from the hub toward the distal end of the catheter, wherein the shaft is more flexible than the hub. In one embodiment the catheter includes an intermediary portion that transitions between the hub and the shaft that has an intermediate flexibility that is less rigid than the hub and more rigid than the shaft. In one embodiment the intermediary portion is a strain relief.

The terms distal and proximal define relative locations of two different features. With respect to a robotic drive the terms distal and proximal are defined by the position of the robotic drive in its intended use relative to a patient. When used to define a relative position, the distal feature is the feature of the robotic drive that is closer to the patient than a proximal feature when the robotic drive is in its intended in-use position. Within a patient, any vasculature landmark further away along the path from the access point is considered more distal than a landmark closer to the access point, where the access point is the point at which the EMD enters the patient. Similarly, the proximal feature is the feature that is farther from the patient than the distal feature when the robotic drive in its intended in-use position. When used to define direction, the distal direction refers to a path on which something is moving or is aimed to move or along which something is pointing or facing from a proximal feature toward a distal feature and/or patient when the robotic drive is in its intended in-use position. The proximal direction is the opposite direction of the distal direction.

The term longitudinal axis of a member (e.g., an EMD or other element in the catheter-based procedure system) is the direction of orientation going from a proximal portion of the member to a distal portion of the member. By way of example, the longitudinal axis of a guidewire is the direction of orientation from a proximal portion of the guide wire toward a distal portion of the guidewire even though the guidewire may be non-linear in the relevant portion. The term axial movement of a member refers to translation of the member along the longitudinal axis of the member. When a distal end of an EMD is axially moved in a distal direction along its longitudinal axis into or further into the patient, the EMD is being advanced. When the distal end of an EMD is axially moved in a proximal direction along its longitudinal axis out of or further out of the patient, the EMD is being withdrawn. The term rotational movement of a member refers to change in angular orientation of the member about the local longitudinal axis of the member. Rotational movement of an EMD corresponds to clockwise or counterclockwise rotation of the EMD about its longitudinal axis due to an applied torque.

The term axial insertion refers to inserting a first member into a second member along the longitudinal axes of the second member. The term lateral insertion refers to inserting a first member into a second member along a direction in a plane perpendicular to the longitudinal axis of the second member. This can also be referred to as radial loading or side loading. The term pinch refers to releasably fixing an EMD to a member such that the EMD and member move together when the member moves. The term unpinch refers to releasing the EMD from a member such that the EMD and member move independently when the member moves. The term clamp refers to releasably fixing an EMD to a member such that the EMD's movement is constrained with respect to the member. The member can be fixed with respect to a global coordinate system or with respect to a local coordinate system. The term unclamp refers to releasing the EMD from the member such that the EMD can move independently.

The term grip refers to the application of a force or torque to an EMD by a drive mechanism that causes motion of the EMD without slip in at least one degree of freedom. The term ungrip refers to the release of the application of force or torque to the EMD by a drive mechanism such that the position of the EMD is no longer constrained. In one example, an EMD gripped between two tires will rotate about its longitudinal axis when the tires move longitudinally relative to one another. The rotational movement of the EMD is different than the movement of the two tires. The position of an EMD that is gripped is constrained by the drive mechanism. The term buckling refers to the tendency of a flexible EMD when under axial compression to bend away from the longitudinal axis or intended path along which it is being advanced. In one embodiment axial compression occurs in response to resistance from being navigated in the vasculature. The distance an EMD may be driven along its longitudinal axis without support before the EMD buckles is referred to herein as the device buckling distance. The device buckling distance is a function of the device's stiffness, geometry (including but not limited to diameter), and force being applied to the EMD. Buckling may cause the EMD to form an arcuate portion different than the intended path. Kinking is a case of buckling in which deformation of the EMD is non-elastic resulting in a permanent set.

The terms top, up, upper, and above refer to the general direction away from the direction of gravity and the terms bottom, down, lower, and below refer to the general direction in the direction of gravity. The term inwardly refers to the inner portion of a feature. The term outwardly refers to the outer portion of a feature. The term front refers to the side of the robotic drive (or an element of the robotic drive or other element of the catheter procedure system) that faces a bedside user and away from the positioning system, such as an articulating arm. The term rear refers to the side of the robotic drive (or an element of the robotic drive or other element of the catheter procedure system) that is closest to the positioning system, such as the articulating arm. The term sterile interface refers to an interface or boundary between a sterile and non-sterile unit. For example, a cassette may be a sterile interface between the robotic drive and at least one EMD. The term sterilizable unit refers to an apparatus that is capable of being sterilized (free from pathogenic microorganisms). This includes, but is not limited to, a cassette, consumable unit, drape, device adaptor, and sterilizable drive modules/units (which may include electromechanical components). Sterilizable Units may come into contact with the patient, other sterile devices, or anything else placed within the sterile field of a medical procedure.

The term on-device adaptor refers to sterile apparatus capable of releasably pinching an EMD to provide a driving interface. For example, the on-device adaptor is also known as an end-effector or EMD capturing device. In one non-limiting embodiment, the on-device adaptor is a collet that is operatively controlled robotically to rotate the EMD about its longitudinal axis, to pinch and/or unpinch the EMD to the collet, and/or to translate the EMD along its longitudinal axis. In one embodiment the on-device adaptor is a hub-drive mechanism such as a driven gear located on the hub of an EMD.

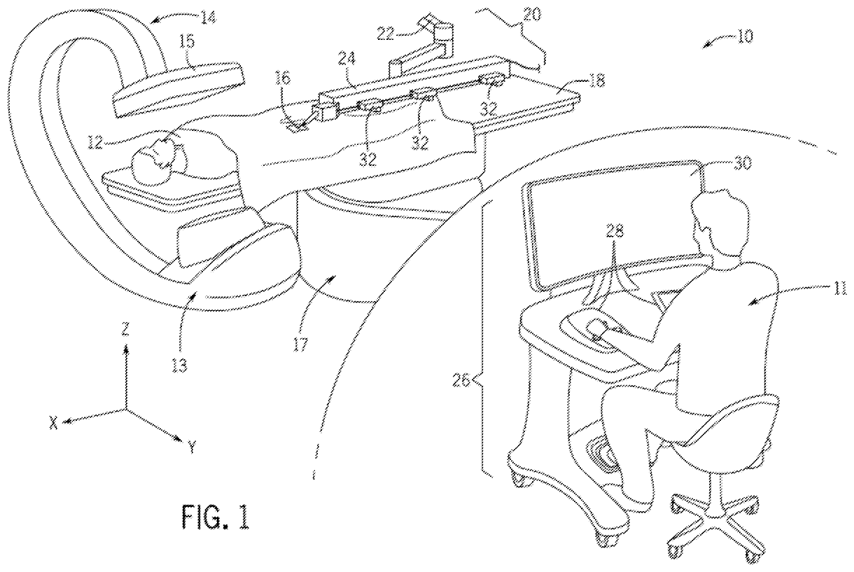

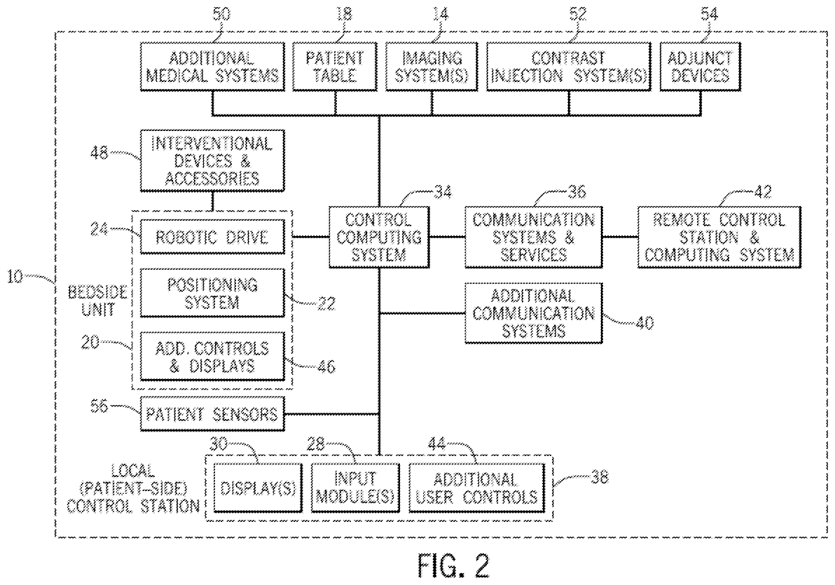

FIG. 1 is a perspective view of an exemplary catheter-based procedure system 10 in accordance with an embodiment. Catheter-based procedure system 10 may be used to perform catheter-based medical procedures, e.g., percutaneous intervention procedures such as a percutaneous coronary intervention (PCI) (e.g., to treat STEMI), a neurovascular interventional procedure (NVI) (e.g., to treat an emergent large vessel occlusion (ELVO)), peripheral vascular intervention procedures (PVI) (e.g., for critical limb ischemia (CLI), etc.). Catheter-based medical procedures may include diagnostic catheterization procedures during which one or more catheters or other elongated medical devices (EMDs) are used to aid in the diagnosis of a patient's disease. For example, during one embodiment of a catheter-based diagnostic procedure, a contrast media is injected onto one or more arteries through a catheter and an image of the patient's vasculature is taken. Catheter-based medical procedures may also include catheter-based therapeutic procedures (e.g., angioplasty, stent placement, treatment of peripheral vascular disease, clot removal, arterial venous malformation therapy, treatment of aneurysm, etc.) during which a catheter (or other EMD) is used to treat a disease. Therapeutic procedures may be enhanced by the inclusion of adjunct devices 54 (shown in FIG. 2) such as, for example, intravascular ultrasound (IVUS), optical coherence tomography (OCT), fractional flow reserve (FFR), etc. It should be noted, however, that one skilled in the art would recognize that certain specific percutaneous intervention devices or components (e.g., type of guidewire, type of catheter, etc.) may be selected based on the type of procedure that is to be performed. Catheter-based procedure system 10 can perform any number of catheter-based medical procedures with minor adjustments to accommodate the specific percutaneous intervention devices to be used in the procedure.

Catheter-based procedure system 10 includes, among other elements, a bedside unit 20 and a control station 26. Bedside unit 20 includes a robotic drive 24 and a positioning system 22 that are located adjacent to a patient 12. Patient 12 is supported on a patient table 18. The positioning system 22 is used to position and support the robotic drive 24. The positioning system 22 may be, for example, a robotic arm, an articulated arm, a holder, etc. The positioning system 22 may be attached at one end to, for example, a rail on the patient table 18, a base, or a cart. The other end of the positioning system 22 is attached to the robotic drive 24. The positioning system 22 may be moved out of the way (along with the robotic drive 24) to allow for the patient 12 to be placed on the patient table 18. Once the patient 12 is positioned on the patient table 18, the positioning system 22 may be used to situate or position the robotic drive 24 relative to the patient 12 for the procedure. In an embodiment, patient table 18 is operably supported by a pedestal 17, which is secured to the floor and/or earth. Patient table 18 is able to move with multiple degrees of freedom, for example, roll, pitch, and yaw, relative to the pedestal 17. Bedside unit 20 may also include controls and displays 46 (shown in FIG. 2). For example, controls and displays may be located on a housing of the robotic drive 24.

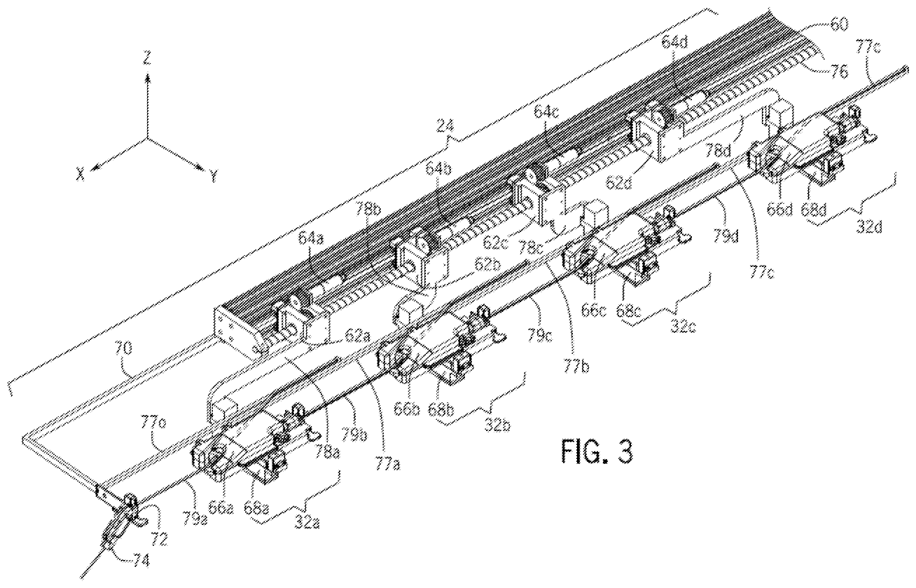

Generally, the robotic drive 24 may be equipped with the appropriate percutaneous interventional devices and accessories 48 (shown in FIG. 2) (e.g., guidewires, various types of catheters including balloon catheters, stent delivery systems, stent retrievers, embolization coils, liquid embolics, aspiration pumps, device to deliver contrast media, medicine, hemostasis valve adaptors, syringes, stopcocks, inflation device, etc.) to allow the user or operator 11 to perform a catheter-based medical procedure via a robotic system by operating various controls such as the controls and inputs located at the control station 26. Bedside unit 20, and in particular robotic drive 24, may include any number and/or combination of components to provide bedside unit 20 with the functionality described herein. A user or operator 11 at control station 26 is referred to as the control station user or control station operator and referred to herein as user or operator. A user or operator at bedside unit 20 is referred to as bedside unit user or bedside unit operator. The robotic drive 24 includes a plurality of device modules 32a-d mounted to a rail or linear member 60 (shown in FIG. 3). The rail or linear member 60 guides and supports the device modules. Each of the device modules 32a-d may be used to drive an EMD such as a catheter or guidewire. For example, the robotic drive 24 may be used to automatically feed a guidewire into a diagnostic catheter and into a guide catheter in an artery of the patient 12. One or more devices, such as an EMD, enter the body (e.g., a vessel) of the patient 12 at an insertion point 16 via, for example, an introducer sheath.

Bedside unit 20 is in communication with control station 26, allowing signals generated by the user inputs of control station 26 to be transmitted wirelessly or via hardwire to bedside unit 20 to control various functions of bedside unit 20. As discussed below, control station 26 may include a control computing system 34 (shown in FIG. 2) or be coupled to the bedside unit 20 through a control computing system 34. Bedside unit 20 may also provide feedback signals (e.g., loads, speeds, operating conditions, warning signals, error codes, etc.) to control station 26, control computing system 34 (shown in FIG. 2), or both. Communication between the control computing system 34 and various components of the catheter-based procedure system 10 may be provided via a communication link that may be a wireless connection, cable connections, or any other means capable of allowing communication to occur between components. Control station 26 or other similar control system may be located either at a local site (e.g., local control station 38 shown in FIG. 2) or at a remote site (e.g., remote control station and computer system 42 shown in FIG. 2). Catheter procedure system 10 may be operated by a control station at the local site, a control station at a remote site, or both the local control station and the remote control station at the same time. At a local site, user or operator 11 and control station 26 are located in the same room or an adjacent room to the patient 12 and bedside unit 20. As used herein, a local site is the location of the bedside unit 20 and a patient 12 or subject (e.g., animal or cadaver) and the remote site is the location of a user or operator 11 and a control station 26 used to control the bedside unit 20 remotely. A control station 26 (and a control computing system) at a remote site and the bedside unit 20 and/or a control computing system at a local site may be in communication using communication systems and services 36 (shown in FIG. 2), for example, through the Internet. In an embodiment, the remote site and the local (patient) site are away from one another, for example, in different rooms in the same building, different buildings in the same city, different cities, or other different locations where the remote site does not have physical access to the bedside unit 20 and/or patient 12 at the local site.

Control station 26 generally includes one or more input modules 28 configured to receive user inputs to operate various components or systems of catheter-based procedure system 10. In the embodiment shown, control station 26 allows the user or operator 11 to control bedside unit 20 to perform a catheter-based medical procedure. For example, input modules 28 may be configured to cause bedside unit 20 to perform various tasks using percutaneous intervention devices (e.g., EMDs) interfaced with the robotic drive 24 (e.g., to advance, retract, or rotate a guidewire, advance, retract or rotate a catheter, inflate or deflate a balloon located on a catheter, position and/or deploy a stent, position and/or deploy a stent retriever, position and/or deploy a coil, inject contrast media into a catheter, inject liquid embolics into a catheter, inject medicine or saline into a catheter, aspirate on a catheter, or to perform any other function that may be performed as part of a catheter-based medical procedure). Robotic drive 24 includes various drive mechanisms to cause movement (e.g., axial and rotational movement) of the components of the bedside unit 20 including the percutaneous intervention devices.

In one embodiment, input modules 28 may include one or more touch screens, joysticks, scroll wheels, and/or buttons. In addition to input modules 28, the control station 26 may use additional user controls 44 (shown in FIG. 2) such as foot switches and microphones for voice commands, etc. Input modules 28 may be configured to advance, retract, or rotate various components and percutaneous intervention devices such as, for example, a guidewire, and one or more catheters or microcatheters. Buttons may include, for example, an emergency stop button, a multiplier button, device selection buttons and automated move buttons. When an emergency stop button is pushed, the power (e.g., electrical power) is shut off or removed to bedside unit 20. When in a speed control mode, a multiplier button acts to increase or decrease the speed at which the associated component is moved in response to a manipulation of input modules 28. When in a position control mode, a multiplier button changes the mapping between input distance and the output commanded distance. Device selection buttons allow the user or operator 11 to select which of the percutaneous intervention devices loaded into the robotic drive 24 are controlled by input modules 28. Automated move buttons are used to enable algorithmic movements that the catheter-based procedure system 10 may perform on a percutaneous intervention device without direct command from the user or operator 11. In one embodiment, input modules 28 may include one or more controls or icons (not shown) displayed on a touch screen (that may or may not be part of a display 30), that, when activated, causes operation of a component of the catheter-based procedure system 10. Input modules 28 may also include a balloon or stent control that is configured to inflate or deflate a balloon and/or deploy a stent. Each of the input modules 28 may include one or more buttons, scroll wheels, joysticks, touch screen, etc. that may be used to control the particular component or components to which the control is dedicated. In addition, one or more touch screens may display one or more icons (not shown) related to various portions of input modules 28 or to various components of catheter-based procedure system 10.

Control station 26 may include a display 30. In other embodiments, the control station 26 may include two or more displays 30. Display 30 may be configured to display information or patient specific data to the user or operator 11 located at control station 26. For example, display 30 may be configured to display image data (e.g., X-ray images, MRI images, CT images, ultrasound images, etc.), hemodynamic data (e.g., blood pressure, heart rate, etc.), patient record information (e.g., medical history, age, weight, etc.), lesion or treatment assessment data (e.g., IVUS, OCT, FFR, etc.). In addition, display 30 may be configured to display procedure specific information (e.g., procedural checklist, recommendations, duration of procedure, catheter or guidewire position, volume of medicine or contrast agent delivered, etc.). Further, display 30 may be configured to display information to provide the functionalities associated with control computing system 34 (shown in FIG. 2). Display 30 may include touch screen capabilities to provide some of the user input capabilities of the system.

Catheter-based procedure system 10 also includes an imaging system 14. Imaging system 14 may be any medical imaging system that may be used in conjunction with a catheter based medical procedure (e.g., non-digital X-ray, digital X-ray, CT, MRI, ultrasound, etc.). In an exemplary embodiment, imaging system 14 is a digital X-ray imaging device that is in communication with control station 26. In one embodiment, imaging system 14 may include a C-arm (shown in FIG. 1) that allows imaging system 14 to partially or completely rotate around patient 12 in order to obtain images at different angular positions relative to patient 12 (e.g., sagittal views, caudal views, anterior-posterior views, etc.). In one embodiment imaging system 14 is a fluoroscopy system including a C-arm having an X-ray source 13 and a detector 15, also known as an image intensifier.

Imaging system 14 may be configured to take X-ray images of the appropriate area of patient 12 during a procedure. For example, imaging system 14 may be configured to take one or more X-ray images of the head to diagnose a neurovascular condition. Imaging system 14 may also be configured to take one or more X-ray images (e.g., real time images) during a catheter-based medical procedure to assist the user or operator 11 of control station 26 to properly position a guidewire, guide catheter, microcatheter, stent retriever, coil, stent, balloon, etc. during the procedure. The image or images may be displayed on display 30. For example, images may be displayed on display 30 to allow the user or operator 11 to accurately move a guide catheter or guidewire into the proper position.

In order to clarify directions, a rectangular coordinate system is introduced with X, Y, and Z axes. The positive X axis is oriented in a longitudinal (axial) distal direction, that is, in the direction from the proximal end to the distal end, stated another way from the proximal to distal direction. The Y and Z axes are in a transverse plane to the X axis, with the positive Z axis oriented up, that is, in the direction opposite of gravity, and the Y axis is automatically determined by right-hand rule.

FIG. 2 is a block diagram of catheter-based procedure system 10 in accordance with an exemplary embodiment. Catheter-procedure system 10 may include a control computing system 34. Control computing system 34 may physically be, for example, part of control station 26 (shown in FIG. 1). Control computing system 34 may generally be an electronic control unit suitable to provide catheter-based procedure system 10 with the various functionalities described herein. For example, control computing system 34 may be an embedded system, a dedicated circuit, a general-purpose system programmed with the functionality described herein, etc. Control computing system 34 is in communication with bedside unit 20, communications systems and services 36 (e.g., Internet, firewalls, cloud services, session managers, a hospital network, etc.), a local control station 38, additional communications systems 40 (e.g., a telepresence system), a remote control station and computing system 42, and patient sensors 56 (e.g., electrocardiogram (ECG) devices, electroencephalogram (EEG) devices, blood pressure monitors, temperature monitors, heart rate monitors, respiratory monitors, etc.). The control computing system is also in communication with imaging system 14, patient table 18, additional medical systems 50, contrast injection systems 52 and adjunct devices 54 (e.g., IVUS, OCT, FFR, etc.). The bedside unit 20 includes a robotic drive 24, a positioning system 22 and may include additional controls and displays 46. As mentioned above, the additional controls and displays may be located on a housing of the robotic drive 24. Interventional devices and accessories 48 (e.g., guidewires, catheters, etc.) interface to the bedside system 20. In an embodiment, interventional devices and accessories 48 may include specialized devices (e.g., IVUS catheter, OCT catheter, FFR wire, diagnostic catheter for contrast, etc.) which interface to their respective adjunct devices 54, namely, an IVUS system, an OCT system, and FFR system, etc.

In various embodiments, control computing system 34 is configured to generate control signals based on the user's interaction with input modules 28 (e.g., of a control station 26 (shown in FIG. 1) such as a local control station 38 or a remote control station 42) and/or based on information accessible to control computing system 34 such that a medical procedure may be performed using catheter-based procedure system 10. The local control station 38 includes one or more displays 30, one or more input modules 28, and additional user controls 44. The remote control station and computing system 42 may include similar components to the local control station 38. The remote 42 and local 38 control stations can be different and tailored based on their required functionalities. The additional user controls 44 may include, for example, one or more foot input controls. The foot input control may be configured to allow the user to select functions of the imaging system 14 such as turning on and off the X-ray and scrolling through different stored images. In another embodiment, a foot input device may be configured to allow the user to select which devices are mapped to scroll wheels included in input modules 28. Additional communication systems 40 (e.g., audio conference, video conference, telepresence, etc.) may be employed to help the operator interact with the patient, medical staff (e.g., angio-suite staff), and/or equipment in the vicinity of the bedside.

Catheter-based procedure system 10 may be connected or configured to include any other systems and/or devices not explicitly shown. For example, catheter-based procedure system 10 may include image processing engines, data storage and archive systems, automatic balloon and/or stent inflation systems, medicine injection systems, medicine tracking and/or logging systems, user logs, encryption systems, systems to restrict access or use of catheter-based procedure system 10, etc.

As mentioned, control computing system 34 is in communication with bedside unit 20 which includes a robotic drive 24, a positioning system 22 and may include additional controls and displays 46, and may provide control signals to the bedside unit 20 to control the operation of the motors and drive mechanisms used to drive the percutaneous intervention devices (e.g., guidewire, catheter, etc.). The various drive mechanisms may be provided as part of a robotic drive 24. FIG. 3 is a perspective view of a robotic drive for a catheter-based procedure system 10 in accordance with an embodiment. In FIG. 3, a robotic drive 24 includes multiple device modules 32a-d coupled to a linear member 60. Each device module 32a-d is coupled to the linear member 60 via a stage 62a-d moveably mounted to the linear member 60. A device module 32a-d may be connected to a stage 62a-d using a connector such as an offset bracket 78a-d. In another embodiment, the device module 32a-d is directly mounted to the stage 62a-d. Each stage 62a-d may be independently actuated to move linearly along the linear member 60. Accordingly, each stage 62a-d (and the corresponding device module 32a-d coupled to the stage 62a-d) may independently move relative to each other and the linear member 60. A drive mechanism is used to actuate each stage 62a-d. In the embodiment shown in FIG. 3, the drive mechanism includes independent stage translation motors 64a-d coupled to each stage 62a-d and a stage drive mechanism 76, for example, a lead screw via a rotating nut, a rack via a pinion, a belt via a pinion or pulley, a chain via a sprocket, or the stage translation motors 64a-d may be linear motors themselves. In some embodiments, the stage drive mechanism 76 may be a combination of these mechanisms, for example, each stage 62a-d could employ a different type of stage drive mechanism. In an embodiment where the stage drive mechanism is a lead screw and rotating nut, the lead screw may be rotated and each stage 62a-d may engage and disengage from the lead screw to move, e.g., to advance or retract. In the embodiment shown in FIG. 3, the stages 62a-d and device modules 32a-d are in a serial drive configuration.

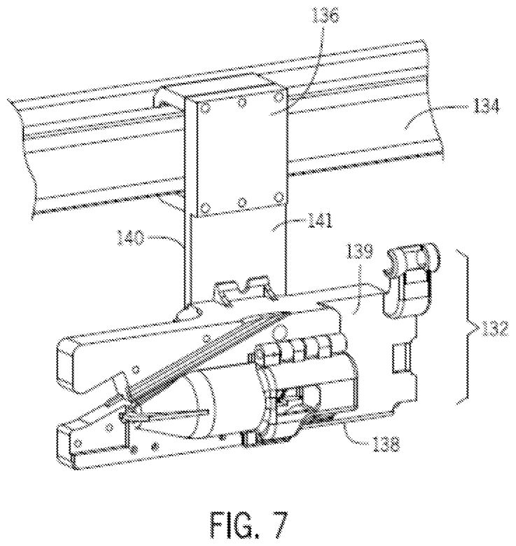

Each device module 32a-d includes a drive module 68a-d and a cassette 66a-d mounted on and coupled to the drive module 68a-d. In the embodiment shown in FIG. 3, each cassette 66a-d is mounted to the drive module 68a-d in an orientation such that the cassette 66a-d is mounted on a drive module 68a-d by moving the cassette 66a-d in a vertical direction down onto the drive module 66a-d. A top face or side of the cassette 66a-d is parallel to a top face or side (i.e., a mounting surface) of the drive module 68a-d when the cassette 66a-d is mounted on the drive module 68a-d. As used herein, the mounting orientation shown in FIG. 3 is referred to as a horizontal orientation. In other embodiments, each cassette 66a-d may be mounted to the drive module 68a-d in other mounting orientations. Various mounting orientations are described further below with respect to FIGS. 7-10. Each cassette 66a-d is configured to interface with and support a proximal portion of an EMD (not shown). In addition, each cassette 66a-d may include elements to provide one or more degrees of freedom in addition to the linear motion provided by the actuation of the corresponding stage 62a-d to move linearly along the linear member 60. For example, the cassette 66a-d may include elements that may be used to rotate the EMD when the cassette is coupled to the drive module 68a-d. Each drive module 68a-d includes at least one coupler to provide a drive interface to the mechanisms in each cassette 66a-d to provide the additional degree of freedom. Each cassette 66a-d also includes a channel in which a device support 79a-d is positioned, and each device support 79a-d is used to prevent an EMD from buckling. A support arm 77a. 77b, and 77c is attached to each device module 32a, 32b, and 32c, respectively, to provide a fixed point for support of a proximal end of the device supports 79b, 79c, and 79d, respectively. The robotic drive 24 may also include a device support connection 72 connected to a device support 79, a distal support arm 70 and a support arm 770. Support arm 770 is used to provide a fixed point for support of the proximal end of the distal most device support 79a housed in the distal most device module 32a. In addition, an introducer interface support (redirector) 74 may be connected to the device support connection 72 and an EMD (e.g., an introducer sheath). The configuration of robotic drive 24 has the benefit of reducing volume and weight of the drive robotic drive 24 by using actuators on a single linear member.

To prevent contaminating the patient with pathogens, healthcare staff use aseptic technique in a room housing the bedside unit 20 and the patient 12 or subject (shown in FIG. 1). A room housing the bedside unit 20 and patient 12 may be, for example, a cath lab or an angio suite. Aseptic technique consists of using sterile barriers, sterile equipment, proper patient preparation, environmental controls and contact guidelines. Accordingly, all EMDs and interventional accessories are sterilized and can only be in contact with either sterile barriers or sterile equipment. In an embodiment, a sterile drape (not shown) is placed over the non-sterile robotic drive 24. Each cassette 66a-d is sterilized and acts as a sterile interface between the draped robotic drive 24 and at least one EMD. Each cassette 66a-d can be designed to be sterile for single use or to be re-sterilized in whole or part so that the cassette 66a-d or its components can be used in multiple procedures.

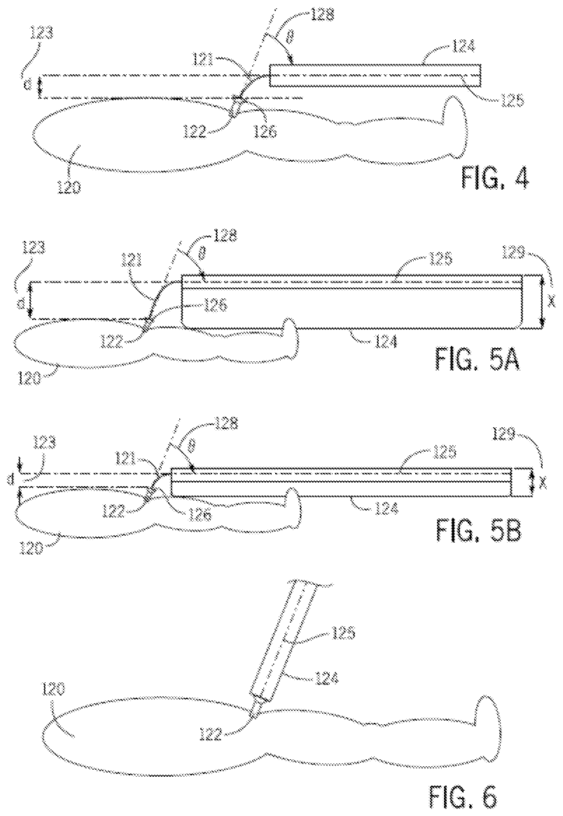

As shown in FIG. 1, one or more EMDs may enter the body of a patient (e.g., a vessel) at an insertion point 16 using, for example, an introducer and introducer sheath. The introducer sheath typically orients at an angle, usually less than 45 degrees, to the axis of the vessel in a patient 120 (shown in FIGS. 4-6). Any height difference between where the EMD enters the body (the introducer sheath's proximal opening 126 shown in FIG. 4) and the longitudinal drive axis of the robotic drive 124 will directly affect the working length for the elongated medical device. The more an elongated medical device needs to compensate for differences in displacement and angle, the less the elongated medical device will be able to enter the body when the robotic drive is at its maximum distal (forward) position. It is beneficial to have a robotic drive that is at the same height and angle as the introducer sheath. FIG. 4 is a diagram illustrating an elongated medical device axis of manipulation and the introductory point into the patient. FIG. 4 shows a height difference (d) 123 between the proximal end 126 of the introducer sheath 122 and the longitudinal device axis and an angular difference (0) 128 between the introducer sheath 122 and the longitudinal device axis 125 of the robotic drive 124. The elongated medical device 121 is constrained on each axis and creates a curve with tangentially aligned end points. The length of this curve represents a length of the elongated medical device 121 that cannot be driven any further forward by the robotic drive 124 and cannot enter the introducer sheath 122 due to the misalignment. A higher angle (0) 128 also leads to higher device friction. In general, lower angular misalignment (0) 128, and linear misalignment d 123 can lead to reduced friction and reduced loss of working length. While FIG. 4 illustrates a simplified example illustrating one linear and one rotational offset, it should be understood that this problem occurs in three dimensions, namely, three linear offsets and three rotational offsets. The thickness of the robotic drive 124 also plays a role in determining the location of the longitudinal device axis 125 relative to the introducer sheath 122.

FIGS. 5a and 5b are diagrams illustrating the effect of the thickness of a drive module, or robotic drive as a whole, on the loss of working length. FIG. 5a shows the location of the longitudinal device axis 125 of a robotic drive 124 relative to the introducer sheath 122, indicated by d 123, when the robotic drive 124 is thick as shown by the distance (X) 129 between an upper surface and a bottom surface of the robotic drive 124. FIG. 5b shows the location of the longitudinal device axis 125 of a robotic drive 124 relative to the introducer sheath 122, indicated by a shorter d 123, when the robotic drive 124 is shallow as shown by the distance (X) 129 between an upper surface and a bottom surface of the robotic drive 124. Reducing the thickness of the robotic drive 124 to get close to the patient and introducer sheath reduces the distance 123 between introducer sheath axis and device axis and reduces the loss of working length of the elongated medical device. FIG. 6 is a diagram illustrating an exemplary orientation to minimize loss of working length. In FIG. 6, the robotic drive is positioned to align the longitudinal device axis 125 of the robotic drive 124 to that of the introducer sheath 122. This eliminates loss of working length due to angular and linear misalignment of the elongated medical device. However, this position for the robotic drive 124 may not be practical due to the length and size of the robotic drive 124. Orienting a robotic drive at a sharp angle also affects the usability by making it difficult to load and unload elongated medical devices, and adjust and handle the robotic drive.

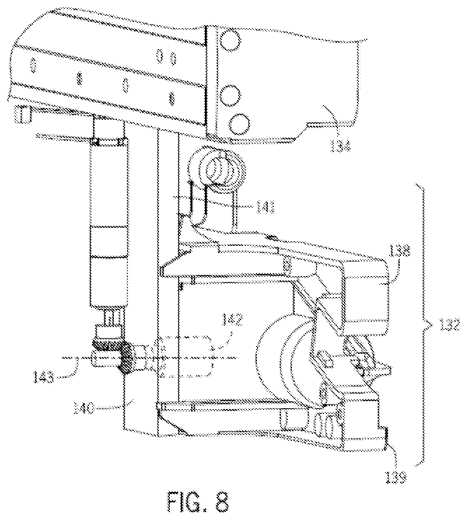

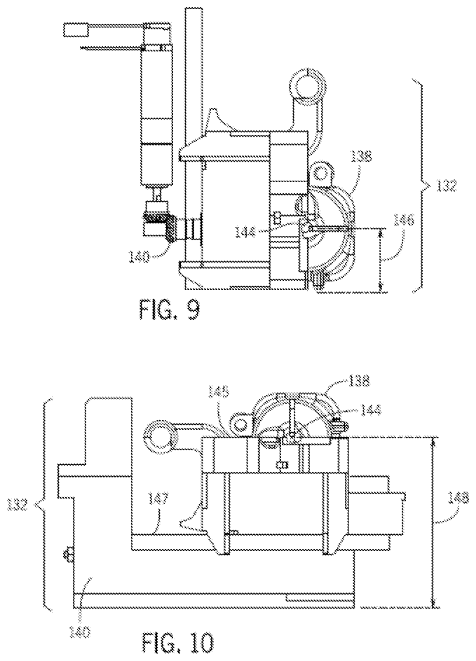

To reduce the distance between the robotic drive and the patient and the distance between the longitudinal device axis of the robotic drive and the introducer sheath, the cassette 66a-d of a device module 32 (shown in FIG. 3) may be mounted to the drive module 68 a-d in an orientation such that the cassette 66a-d is mounted on a drive module 68a-d by moving the cassette 66a-d in a horizontal direction onto the drive module 66a-d. FIG. 7 is a perspective view of a device module with a vertically mounted cassette in accordance with an embodiment and FIG. 8 is a rear perspective view of a device module with a vertically mounted cassette in accordance with an embodiment. In FIGS. 7 and 8, a device module 132 includes a cassette 138 that is mounted to a drive nodule 140 such that front face or side 139 of the cassette 138 is parallel to a front face or side 141 (i.e., a mounting surface) of the drive module 140. As used herein, the mounting orientation shown in FIGS. 7 and 8 is referred to as a vertical orientation. The device module 132 is connected to a stage 136 that is moveably mounted to a rail or linear member 134. The drive module 140 includes a coupler 142 that is used to provide a power interface to the cassette 138 to, for example, rotate an elongated medical device (not shown) positioned in the cassette. The coupler 142 rotates about an axis 143. As mentioned, the cassette 138 is mounted to the drive module 140 by moving the cassette 138 in a horizontal direction onto the mounting surface 141 so that the cassette is coupled to coupler 142 of the drive module 140. By mounting the cassette 138 vertically, the drive module 140 that the cassette 138 attaches to located off to the side and no longer positioned between the cassette 138 and the patient. FIG. 9 is a front view of a distal end of a device module with a vertically mounted cassette in accordance with an embodiment. In FIG. 9, a distance 146 between the device axis of the elongated medical device 144 and the bottom surface of the device module 132 is shown. The vertical mounting orientation of the cassette 138 eliminates the need for the drive module 140 to be placed under the device axis and between the elongated medical device 144 and the patient. Rather, only a portion of the cassette 138 is positioned between the elongated medical device 138 and the patient. Vertically mounting the cassette 138 also reduces the distance 146 between the elongated medical device and bottom surface of the device module 132 which allows the robotic drive to get closer to the patient and reduces loss of working length in an elongated medical device. By comparison, FIG. 10 is a front view of a distal end of a device module with a horizontally mounted cassette in accordance with an embodiment. In FIG. 10, a device module 132 is shown where the cassette 138 is horizontally mounted to a drive module 140. A top face or side 145 of the cassette 138 is parallel to a top face or side 147 (i.e., a mounting surface) of the drive module 140 when the cassette 138 is mounted on the drive module 140. The drive module 140 is under or below the cassette 138 and increases the distance 148 between the device axis of the elongated medical device 144 and the bottom surface of the device module 132. This can prevent the device axis from being as close to the introducer (not shown) as possible. A drive module 140 positioned under the cassette 138 may also interfere with the patient. In various other embodiment, a cassette may be mounted to the drive module at any angle. In yet another embodiment, the cassette may be mounted horizontally on an underside of the drive module to eliminate the need for a drive module between the device axis and the patient.

An EMD (e.g., a catheter) in a cassette may be connected to various tubing to, for example, supply a saline drip, to allow for contrast injection, to allow for aspiration, etc. In an embodiment, a catheter may be coupled to a hemostasis valve (e.g., a rotating hemostasis valve) which has a side port that may be coupled to the tubing (e.g., releasably coupled or permanently coupled). In some systems for fluid management, a closed system utilizing a manifold may be used to provide connections to all of the necessary fluid lines. In a closed system, all the necessary fluid lines (e.g., saline, contrast, waste bag) are connected through a series of stopcocks to side ports of a manifold. A syringe is connected to the proximal end of the manifold and tubing is connected at the distal end of the manifold. The other end of the tubing is connected to a side port of the hemostasis valve which is in fluid communication with the catheter. Once set up, no connections are removed to ensure that the air does not enter the system. Accordingly, a closed system requires multiple fluid lines to be dedicated to a manifold to inject fluid into or aspirate fluid from a catheter. If there is more than one catheter that requires a fluid connection in a system, a closed system with a manifold and all the required fluid lines would need to be set up for each catheter. For an interventional procedure requiring multiple catheters, setting up a closed system for each catheter is burdensome and not necessary.

In a robotic drive that manipulates EMDs linearly, the hemostasis valve and any tubing coupled to the hemostasis valve translate with the catheter as it is advanced and retracted by the robotic drive during a procedure. During movement of the catheter, the tubing may get hung up or snagged on one or more elements of the robotic drive. The possibility of a tube catching may be higher in a robotic drive such as described above because the user is typically not at the bedside viewing and managing tubes, but rather operating the robotic drive from a control station at a local or remote site. A caught or snagged tube could result in resistance to the movement of the robotic drive, a tube breaking, a connection to a tube coming undone, or the hemostasis valve and catheter being yanked out of the robotic system. Accordingly, it would be advantageous to account for tubing connections and provide an apparatus for management of fluid connections to prevent unintended catheter motion or damage if the tubing is snagged or caught during operation of the robotic drive. In addition, it would be advantageous to provide an open system for fluid management.

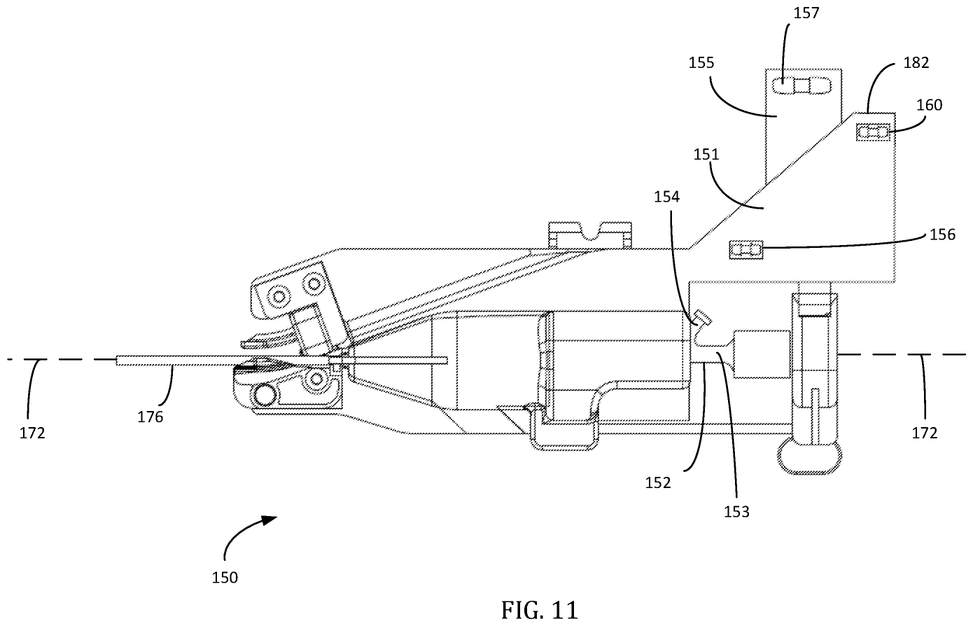

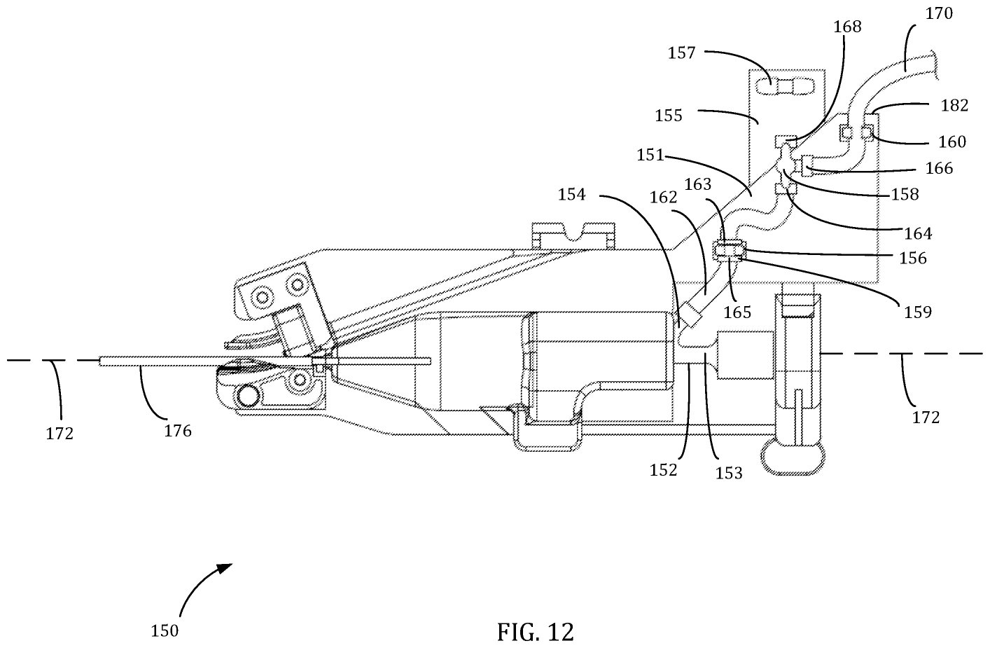

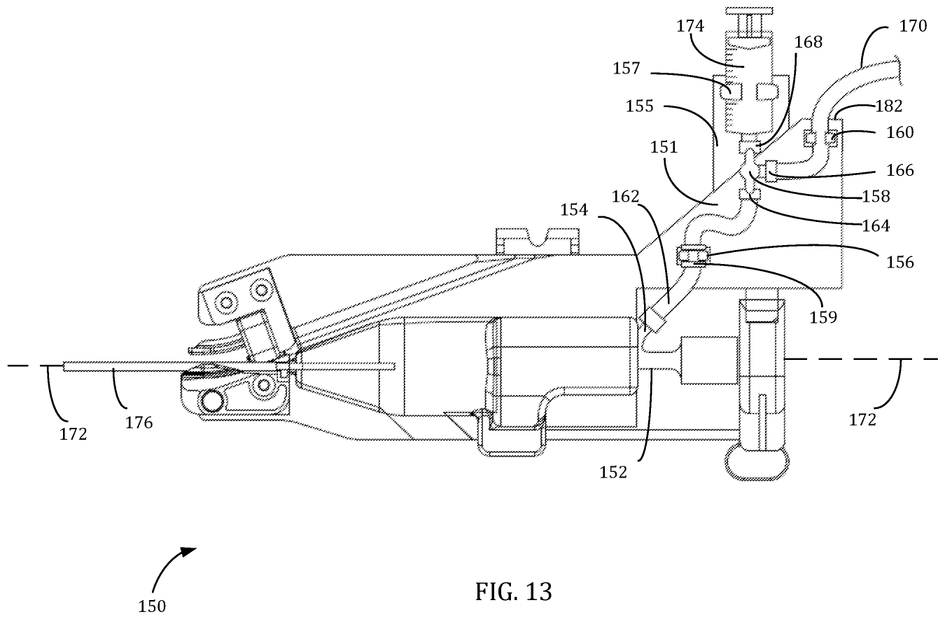

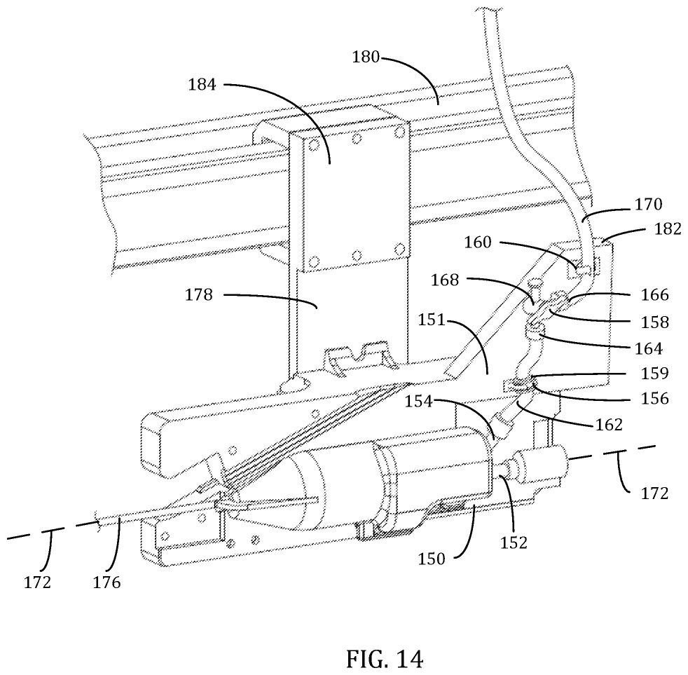

As mentioned, a catheter positioned in a cassette may be coupled to tubing for fluids via a hemostasis valve. FIG. 11 is a front view of a cassette including fluid management elements in accordance with an embodiment and FIG. 12 is a front view of an apparatus for fluid management in accordance with an embodiment. In FIG. 11, a hemostasis valve 152 (e.g., a rotating hemostasis valve) and catheter 176 are positioned in a housing 151 of a cassette 150. Catheter 176 defines a longitudinal device axis 172 of the cassette 150. The hemostasis valve 152 is coupled to the catheter 176. Hemostasis valve 152 includes a base 153 with a lumen that may be used to receive other EMDs, for example, an EMD from another more proximal cassette in the robotic drive (e.g., robotic drive 24 described above with respect to FIGS. 1 and 3). In an embodiment, a distal end (not shown) of the base 153 may include a rotating connector (not shown), for example, a rotating luer connector, that is rotatably connected to a distal end of the base 153. In an embodiment, an external surface of the rotating luer connector includes a gear (not shown) that may be driven by, for example, a robotic drive. Hemostasis valve 150 also includes a side port 154 that may be used to provide a connection to tubing for fluids to go into and out of the catheter 176. In an embodiment, the side port 154 is turned so the open end is pointed up towards the top side of the cassette 150 when positioned in a cassette 150 that is configured to be mounted vertically on a drive module (e.g., cassette 150 shown in FIGS. 13 and 14). A support 155 is connected to the cassette housing 151 and includes a connector 157. The connector 157 is configured to receive a syringe as discussed further below with respect to FIG. 13.

Cassette 150 also includes a first tube connection point 156 and a second tube connection point 160. The first tube connection point 156 is positioned on the housing 151 at a location above the longitudinal device axis 172. The second tube connection point 160 is positioned on the housing 151 proximate to a top edge 182 of the cassette housing 151 and above the first tube connection point 156 and the longitudinal device axis 172. While the first tube connection point 156 and the second tube connection point 160 are shown positioned in a horizontal direction, in various other embodiments the first tube connection point 156 and the second tube connection point 160 may be positioned in a vertical direction or at different angles. The first tube connection point 156 is configured to receive a first tube 162 as shown in FIG. 12. Referring now to FIG. 12, one end of the first tube 162 is connected to the side port 154 of the hemostasis valve 152 and the other end of the first tube 162 is connected to a valve, for example, the three-way stopcock valve 158. In various embodiments, the first tube 162 may be releasably coupled to the side port 154 or the first tube 162 may be permanently coupled (e.g., bonded) to the side port 154. The three-way stopcock valve 168 has a first port 164, a second port 166 and a third port 168.

In the embodiment of FIG. 12, the first tube 162 is connected to a first port 164 of the stopcock valve 158. In various embodiments, the first tube 162 may be releasably coupled to the first port 164 of the stopcock 158 or the first tube 162 may be permanently coupled (e.g., bonded) to the first port 164 of the stopcock 158. The stopcock 158 is not hard mounted to the cassette 150, but is left loose. The first tube connection point 156 is connected to the first tube 162 along the length of the first tube 162. The first tube connection point may be, for example, a clip. The second tube connection point 160 is configured to receive a second tube 170. One end of the second tube 170 may be connected to a second port 166 of the stopcock valve 158. In various embodiments, the second tube 170 may be releasably coupled to the second port 166 of the stopcock 158 or the second tube 170 may be permanently coupled (e.g., bonded) to the second port 166 of the stopcock 158. The other end of the second tube 170 may be connected to a fluid source (not shown) as discussed further below. The second tube 170 is in fluid communication with the first tube 162 and hemostasis valve 152 via the stopcock valve 158.

The first tube connection point 156 is configured to anchor the first tube 162 to the cassette housing 151 (e.g., to prevent radial and axial movement of the first tube 162) and to provide strain relief for the first tube 162 and the hemostasis valve 152. In an embodiment, the first tube 162 may include a collar 159 that engages with the first tube connection point 156 and is configured to prevent axial movement of the first tube 162. In an embodiment, the collar 159 is on an external surface of the first tube 162 and includes an upper flange 163 and a lower flange 165. The first tube connection point 156 in configured to prevent snags or catches of the first tube 162 or the second tube 170 from pulling on the hemostasis valve 152 or pulling the hemostasis valve 151 out of the cassette 150.

FIG. 13 is a front view of an apparatus for fluid management in accordance with an embodiment. As mentioned above, the stopcock valve 158 is not hard mounted to the cassette 150, but is left loose which allows the user to easily and comfortably manipulate the stopcock valve 158 for de-bubbling or connecting a syringe to the stopcock valve 158. In FIG. 13, a syringe 174 is connected to a third port 168 of the stopcock valve 158. The syringe 174 may be used to, for example, inject contrast agent, injecting saline or for aspiration. In an embodiment, the syringe 174 may be positioned on a support 155 and in a connector 157. Support 155 is coupled to the cassette housing 151. The connector 157 may be, for example, a clip or other connection mechanism. The support 155 and connector 157 are configure to provide support to the syringe 174 and prevent movement of the syringe 174 while it is connected to the stopcock valve 158, for example, during a procedure. In addition, the support 155 and connector 157 hold the syringe 174 in place as the cassette 150 (and an associated drive module (not shown)) are moved linearly along the linear member 60 (shown in FIG. 3) during a procedure.

As mentioned, the second tube 170 may be used to provide a fluid (e.g., saline) to the first tube 162 and the hemostasis valve 152 from a fluid source, for example, a pressurized saline bag. In an embodiment, a fluid, such as saline, may be use to flush the lumen of the catheter 176 while the catheter is in use to make sure that blood is not stagnant inside the lumen which may result in clotting. The pressurized bag, or other fluid source, is typically located on a rear or non-operative side of the patient table. The second tube 170 may be draped over the robotic drive to reach the cassette as shown in FIG. 14. FIG. 14 is a perspective views of a device module with a vertically amounted cassette and an apparatus for fluid management in accordance with various embodiments. In FIG. 14, the cassette 150 is shown mounted vertically to a drive module 178. The drive module 178 is coupled to a stage 184 which is moveably coupled to a rail 180. A stopcock valve 158 is connected to a first tube 162 via a first port 162 and the second tube 170 via a second port 166. A third port 168 may be connect to, for example, a syringe (not shown). The first port 164, second port 166 and third port 168 each have a lumen to allow fluid communication with an attached tube (or fluid line) or device (e.g., a syringe). The second tube 170 is positioned in the second tube connection point 160 which is located proximate to a top edge 182 of the cassette housing 151. The second tube connection point 160 directs the second tube 170 up and away from the longitudinal device axis 172 to prevent the second tube 170 from being tangled or snagged with elements of the robotic drive (e.g., the support track 79a-d shown in FIG. 3). During the loading and exchange of an EMD such as catheter 176, the hemostasis valve 152 may be removed from the cassette 150. It is desirable to prevent the loose second tube 170 from falling back over the rear side of the drive module when it is disconnected from the hemostasis valve 152. Accordingly, the second tube connection point 160 is also configured to restrain the second tube 170 to keep it from falling away when it is not coupled to the hemostasis valve 152 and the stopcock valve 158. In an embodiment, a distal end of the second tube 170 that is below the second tube connection point 160 may include a shoulder that prevents the second tube 170 from sliding through the second tube connection point 160 and falling when it is not connected to the stopcock valve 158. The second tube connection point 160 may be, for example, a clip or loop. The second tube connection point 160 is also configured to allow the second tube 170 to move or slide axially within the second tube connection point 160. This allows for ease of handling when the second tube 170 is coupled to the hemostasis valve via the stopcock valve 158 and is being manipulated to allow, for example, de-bubbling.

A control computing system as described herein may include a processor having a processing circuit. The processor may include a central purpose processor, application specific processors (ASICs), circuits containing one or more processing components, groups of distributed processing components, groups of distributed computers configured for processing, etc. configured to provide the functionality of module or subsystem components discussed herein. Memory units (e.g., memory device, storage device, etc.) are devices for storing data and/or computer code for completing and/or facilitating the various processes described in the present disclosure. Memory units may include volatile memory and/or non-volatile memory. Memory units may include database components, object code components, script components, and/or any other type of information structure for supporting the various activities described in the present disclosure. According to an exemplary embodiment, any distributed and/or local memory device of the past, present, or future may be utilized with the systems and methods of this disclosure. According to an exemplary embodiment, memory units are communicably connected to one or more associated processing circuit. This connection may be via a circuit or any other wired, wireless, or network connection and includes computer code for executing one or more processes described herein. A single memory unit may include a variety of individual memory devices, chips, disks, and/or other storage structures or systems. Module or subsystem components may be computer code (e.g., object code, program code, compiled code, script code, executable code, or any combination thereof) for conducting each module's respective functions.

This written description used examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. The order and sequence of any process or method steps may be varied or re-sequenced according to alternative embodiments.

Many other changes and modifications may be made to the present invention without departing from the spirit thereof. The scope of these and other changes will become apparent from the appended claims.

We claim:

1. A cassette for use in a robotic drive of a catheter-based procedure system, the cassette comprising:
a housing configured to support a hemostasis valve having a base and a side port, the housing having a longitudinal device axis associated with an elongated medical device;
a first tube connection point positioned on the housing and above the longitudinal device axis, the first tube connection point configured to receive a first tube; and
a second tube connection point positioned proximate to a top edge of the housing and above the first tube connection point and the longitudinal device axis, the second tube connection point configured to receive a second tube.

2. The cassette according to claim 1, wherein the first tube connection point is configured to provide strain relief for the first tube.

3. The cassette according to claim 2, wherein the first tube connection point is a clip.

4. The cassette according to claim 1, wherein the second tube connection point is a clip.

5. The cassette according to claim 1, wherein the second tube connection point is a loop.

6. An apparatus for providing a fluid connection to a cassette for use in a robotic drive of a catheter-based procedure system, the apparatus comprising:
a cassette housing having a longitudinal device axis associated with an elongated medical device;
a hemostasis valve positioned in the cassette housing, the hemostasis valve having a base and a side port;
a first tube connection point positioned on the cassette housing and above the longitudinal device axis;
a first tube coupled to the side port of the hemostasis valve and positioned in the first connection point;
a valve having a plurality of ports wherein one of the plurality of ports is coupled to the first tube;
a second tube connection point positioned proximate to a top edge of the cassette housing and above the first tube connection point and the longitudinal device axis; and
a second tube coupled to one of the plurality of ports of the valve and positioned in the second tube connection point.

7. The apparatus according to claim 6, wherein the first tube connection point is configured to provide strain relief for the first tube.

8. The apparatus according to claim 7, wherein the first tube connection point is a clip.

9. The apparatus according to claim 6, wherein the valve is a stopcock valve.

10. The apparatus according to claim 9, wherein the plurality of ports includes three ports.

11. The apparatus according to claim 6, wherein the second tube connection point is a clip.

12. The apparatus according to claim 6, wherein the second tube connection point is a loop.

13. The apparatus according to claim 6, wherein the second connection point is configured to allow the second tube to move axially.

14. The apparatus according to claim 13, wherein the second tube includes a shoulder at a distal end of the second tube.

15. The apparatus according to claim 6, wherein the second tube is coupled to a fluid source.

16. The apparatus according to claim 15, wherein the fluid source includes saline.

17. The apparatus according to claim 6, wherein the hemostasis valve is a rotating hemostasis valve.

18. The apparatus according to claim 6, wherein the first tube includes a collar that is configured to engage the first tube connection point.
